(19) United States
(12) Patent Application Publication
(10) Pub. No.: US 2025/0010047 A1
(43) Pub. Date: Jan. 9, 2025

(54) BRAIN DISEASE TREATMENT DEVICE, CONNECTOR FOR TREATMENT DEVICE, AND CONNECTOR FIXTURE FOR TREATMENT DEVICE

(71) Applicant: TERUMO KABUSHIKI KAISHA, Tokyo (JP)

(72) Inventors: Takashi KITAOKA, Hadano city (JP); Taishi NIIMI, Hadano city (JP); Takayuki UCHIDA, Hiratsuka city (JP); Yu OSAWA, Ebina-city (JP)

(73) Assignee: TERUMO KABUSHIKI KAISHA, Tokyo (JP)

(21) Appl. No.: 18/893,052

(22) Filed: Sep. 23, 2024

Related U.S. Application Data
(63) Continuation of application No. PCT/JP2023/009221, filed on Mar. 10, 2023.

(30) Foreign Application Priority Data
Mar. 23, 2022 (JP) 2022-047262

(51) Int. Cl. A61M 27/00; A61M 13/00

(52) U.S. Cl. CPC A61M 27/006; A61M 13/003; A61M 2202/0208

(57) ABSTRACT

A brain disease treatment device includes a first tube and a second tube which has an outer diameter smaller than an inner diameter of the first tube, can be arranged in an inner cavity of the first tube, and can move in the inner cavity of the first tube. The first tube is inserted into a subarachnoid space of a patient and discharges cerebrospinal fluid present in the subarachnoid space out of a body of the patient through a space between the inner cavity of the first tube and the outer side of the second tube. The second tube is inserted into the subarachnoid space and injects fluid through an inner cavity of the second tube into the cerebrospinal fluid present in the subarachnoid space. An end portion of the second tube is exposable from an end portion of the first tube in a longitudinal direction of the first tube.

### CROSS-REFERENCES TO RELATED APPLICATIONS

This application is a continuation of International Application No. PCT/JP2023/009221 filed on Mar. 10, 2023, which claims priority to Japanese Application No. 2022-04762 filed on Mar. 23, 2022, the entire content of both of which is incorporated herein by reference.

### TECHNOLOGICAL FIELD

The present disclosure generally relates to a brain disease treatment device used for treatment of a brain disease, a connector for a treatment device connected to the treatment device, and a connector fixture for a treatment device for fixing a connector for a treatment device to a body surface of a patient.

### BACKGROUND DISCUSSION

When cerebral infarction, for example, occurs as a brain disease, a blood flow supplying oxygen to brain cells is blocked, and there is a possibility that the brain cells are damaged. Therefore, when the cerebral infarction occurs, early reperfusion of the blood flow is necessary. However, the percentage of patients who meet conditions for receiving hyperacute treatment at a high evidence level (for example, tissue plasminogen activator (t-PA) administration, mechanical thrombectomy (MT), or the like) currently in clinical practice is relatively low. Therefore, many patients can select only conservative treatment.

As one of treatments for the cerebral infarction, it has been proposed that a

high oxygen solution such as oxygenated cerebrospinal fluid is injected into an intrathecal space of a patient to supply oxygen directly to oxygen-deficient brain cells. U.S. Pat. No. 4,686,085 discloses a method and system for providing early stroke treatment. In the method and system described in U.S. Pat. No. 4,686,085, a nutrient emulsion is recovered from the cisterna magna via a conduit having a distal portion inserted into the vicinity of the cisterna magna. Furthermore, an oxygenated nutrient emulsion is also injected into a lateral ventricle through an injection cannula attached to a skull by a fitting.

The method and system described in U.S. Pat. No. 4,686,085 perform injection of a high oxygen solution and discharge of cerebrospinal fluid at two puncture sites. However, in view of invasion to patients, it is desirable to enable the injection of the high oxygen solution and the discharge of the cerebrospinal fluid at one puncture site.

### SUMMARY

Therefore, it would be desirable to insert one catheter into an intrathecal space of a patient from one puncture site to perform the injection of the high oxygen solution and the discharge of the cerebrospinal fluid. In this case, considering the efficiency in the injection of the high oxygen solution and the discharge of the cerebrospinal fluid, it is also desirable to independently adjust an injection position of the high oxygen solution and a drawing position (that is, a discharge position) of the cerebrospinal fluid in an insertion direction (that is, a longitudinal direction) of the catheter after one catheter is inserted into a living body of the patient.

A brain disease treatment device is disclosed that is capable of performing injection and discharge at one puncture site and capable of independently adjusting an injection position and a discharge position after being inserted into a living body, a connector for a treatment device connected to the treatment device, and a connector fixture for a treatment device for fixing a connector for a treatment device to a body surface of a patient.

A brain disease treatment device according to the present disclosure includes: a first tube; and a second tube which has an outer diameter smaller than an inner diameter of the first tube and is arrangeable in an inner cavity of the first tube and movable in the inner cavity of the first tube, the first tube being inserted into a subarachnoid space of a patient and discharging cerebrospinal fluid present in the subarachnoid space out of a body of the patient through a space between the inner cavity of the first tube and an outer side of the second tube, the second tube being inserted into the subarachnoid space and injecting a fluid through an inner cavity of the second tube into the cerebrospinal fluid present in the subarachnoid space, and the second tube has an end portion being exposable from an end portion of the first tube in a longitudinal direction of the first tube.

With the brain disease treatment device according to the present disclosure, the outer diameter of the second tube is smaller than the inner diameter of the first tube. The second tube can be arranged in the inner cavity of the first tube and moveable in the inner cavity of the first tube. The first tube is inserted into the subarachnoid space of the patient and discharges the cerebrospinal fluid present in the subarachnoid space out of the body of the patient through the space between the inner cavity of the first tube and the outer side of the second tube. The second tube is inserted into the subarachnoid space and injects the fluid through the inner cavity of the second tube into the cerebrospinal fluid present in the subarachnoid space. Here, the end portion of the second tube can be exposed from the end portion of the first tube in the longitudinal direction of the first tube. Therefore, after the first tube and the second tube are inserted into the subarachnoid space of the patient, the second tube can move in the inner cavity of the first tube and be exposed from the end portion of the first tube. With this configuration, the fluid can be injected into the cerebrospinal fluid and the cerebrospinal fluid can be discharged at one puncture site, and an injection position of the second tube for injecting the fluid into the cerebrospinal fluid and a discharge position of the first tube for discharging the cerebrospinal fluid out of the body of the patient can be independently adjusted.

In the brain disease treatment device according to the present disclosure, preferably, a distance in the longitudinal direction between the end portion of the first tube and the end portion of the second tube exposed from the end portion of the first tube is adjustable to a predetermined distance or more.

With the brain disease treatment device according to the present disclosure, the distance in the longitudinal direction between the end portion of the first tube and the end portion of the second tube exposed from the end portion of the first tube can be adjusted to the predetermined distance or more. Therefore, the discharge position of the first tube and the injection position of the second tube can be separated by a predetermined distance or more in the longitudinal direction. Therefore, the fluid injected into the cerebrospinal fluid by the second tube can be prevented from being immediately drawn by the first tube and discharged out of the body of the patient, which makes it possible to efficiently discharge the cerebrospinal fluid and inject the fluid into the cerebrospinal fluid.

In the brain disease treatment device according to the present disclosure, preferably, a cross-sectional area of the space between the inner cavity of the first tube and the outer side of the second tube is within a range of a predetermined ratio with respect to a cross-sectional area of the inner cavity of the second tube in a cross section in a direction perpendicular to the longitudinal direction.

With the brain disease treatment device according to the present disclosure, the cross-sectional area of the space between the inner cavity of the first tube and the outer side of the second tube is within the predetermined ratio with respect to the cross-sectional area of the inner cavity of the second tube in the cross section in the direction perpendicular to the longitudinal direction, which makes it possible to efficiently perform the discharge of the cerebrospinal fluid and the injection while keeping the balance between the discharge of the cerebrospinal fluid and the injection of the fluid into the cerebrospinal fluid.

In the brain disease treatment device according to the present disclosure, preferably, the first tube has a plurality of holes on a side surface of the first tube, the plurality of holes allowing the inner cavity of the first tube to communicate with an outer side of the first tube to communicate with each other, and the plurality of holes are provided at positions different from each other in a circumferential direction of the first tube and separated from each other at substantially equal intervals.

With the brain disease treatment device according to the present disclosure, the first tube draws the cerebrospinal fluid from the outer side of the first tube into the inner cavity of the first tube through the plurality of holes provided on the side surface of the first tube, and discharges the cerebrospinal fluid out of the body of the patient. The plurality of holes of the first tube are provided at the positions different from each other in the circumferential direction of the first tube and separated from each other at substantially equal intervals. Therefore, it is possible to prevent all of the plurality of holes from being blocked by tissue even in a case where the tissue in a living body of the patient adheres to any one of the plurality of holes when the first tube is inserted into the subarachnoid space to draw the cerebrospinal fluid. As a result, it is possible to prevent the first tube from being unable to draw and discharge the cerebrospinal fluid.

In the brain disease treatment device according to the present disclosure, preferably, the plurality of holes are provided at positions different from each other in the longitudinal direction and separated from each other at substantially equal intervals.

With the brain disease treatment device according to the present disclosure, the plurality of holes of the first tube are provided at the positions different from each other in the circumferential direction and the longitudinal direction of the first tube and separated from each other at substantially equal intervals. Therefore, the plurality of holes are arranged in a spiral shape on the side surface of the first tube. Therefore, it is possible to further prevent all of the plurality of holes from being blocked by tissue even in a case where the tissue in a living body of the patient adheres to any one of the plurality of holes when the first tube is inserted into the subarachnoid space to draw the cerebrospinal fluid. As a result, it is possible to further prevent the first tube from being unable to draw and discharge the cerebrospinal fluid.

In the brain disease treatment device according to the present disclosure, preferably, the fluid is a high oxygen solution.

With the brain disease treatment device according to the present disclosure, the high oxygen solution can be injected into the cerebrospinal fluid and discharge the cerebrospinal fluid at one puncture site, and the injection position of the second tube for injecting the high oxygen solution into the cerebrospinal fluid and the discharge position of the first tube for discharging the cerebrospinal fluid out of the body of the patient can be independently adjusted. As a result, when cerebral infarction has occurred, it is possible to adopt an arrangement capable of efficiently delivering the high oxygen solution according to the body shape of the patient and a region where the cerebral infarction has occurred, and thus, damage to brain cells can be suppressed. Furthermore, when the cerebral infarction has occurred, the tubes can be appropriately arranged in accordance with a location of the occurrence, and the high oxygen solution can be efficiently administered to suppress the damage to the brain cells.

A connector for a treatment device according to the present disclosure, the connector being connected to a treatment device, which includes a first tube and a second tube having an outer diameter smaller than an inner diameter of the first tube and being arrangeable in an inner cavity of the first tube and movable in the inner cavity of the first tube, the first tube being inserted into a living body lumen of a patient and discharging a fluid present in the living body lumen out of a body of the patient through a space between the inner cavity of the first tube and an outer side of the second tube, and the second tube being inserted into the living body lumen and injecting a fluid into the living body lumen through an inner cavity of the second tube, the connector including: a first holding portion that has a first close contact portion whose inner diameter is equal to or smaller than an outer diameter of the first tube and holds the first tube inserted from a first insertion port at the first close contact portion; a second holding portion that has a second close contact portion whose inner diameter is equal to or smaller than the outer diameter of the second tube and holds the second tube inserted from a second insertion port at the second close contact portion; and an outlet portion that has a flow path through which a fluid passing through the space between the inner cavity of the first tube and the outer side of the second tube flows, and an outlet of the flow path, the second close contact portion being present on an inner side of the first close contact portion when viewed along an axial direction of either the first tube held by the first holding portion or the second tube held by the second holding portion.

With the connector for a treatment device according to the present disclosure, the first holding portion has the first close contact portion whose inner diameter is equal to or smaller than the outer diameter of the first tube of the treatment device, and holds the first tube of the treatment device inserted from the first insertion port at the first close contact portion. The second holding portion has the second close contact portion whose inner diameter is equal to or smaller than the outer diameter of the second tube of the treatment device, and holds the second tube of the treatment device inserted through the second insertion port at the second close contact portion. Therefore, for example, after the first tube of the treatment device is inserted into a living body of the patient through a spinal needle stuck into the living body of the patient and the spinal needle is removed, the first holding portion can hold the first tube of the treatment device inserted from the first insertion port at the first close contact portion. Furthermore, the second holding portion can hold the second tube of the treatment device inserted through the second insertion port at the second close contact portion.

In accordance with an embodiment, the second close contact portion is present on the inner side of the first close contact portion when viewed along the axial direction of either the first tube of the treatment device held by the first holding portion or the second tube of the treatment device held by the second holding portion. Therefore, the second tube of the treatment device can be easily arranged in the inner cavity of the first tube of the treatment device through the second close contact portion and easily moved in the inner cavity of the first tube of the treatment device. Further, the outlet portion has the flow path through which the fluid passing through the space between the inner cavity of the first tube of the treatment device and the outer side of the second tube of the treatment device flows, and the outlet of the flow path. In this manner, when the first tube of the treatment device is inserted into the living body of the patient using, for example, the spinal needle, the connector for a treatment device according to the present invention can be mounted on the first tube of the treatment device after the spinal needle is removed. Then, after the connector for a treatment device according to the present disclosure is mounted on the first tube of the treatment device, the second tube of the treatment device can be easily placed in the inner cavity of the first tube of the treatment device and can be easily moved in the inner cavity of the first tube of the treatment device.

In the connector for a treatment device according to the present disclosure, preferably, the first tube is fixable inside the first holding portion, the second tube inserted from the second insertion port is insertable into the inner cavity of the first tube fixed to the first holding portion and is capable of penetrating the first tube, and the fluid discharged from a proximal portion of the first tube fixed to the first holding portion passes through the flow path.

With the connector for a treatment device according to the present disclosure, the first tube of the treatment device can be fixed inside the first holding portion. The second tube of the treatment device inserted through the second insertion port can be inserted into the inner cavity of the first tube of the treatment device fixed to the first holding portion and can penetrate the first tube of the treatment device. Then, the fluid discharged from the proximal portion of the first tube of the treatment device fixed to the first holding portion passes through the flow path of the outlet portion. Therefore, when the first tube of the treatment device is inserted into the living body of the patient using, for example, the spinal needle, the connector for a treatment device according to the present disclosure is mounted on the first tube and the second tube of the treatment device after the spinal needle is removed, so that the fluid discharged from the proximal portion of the first tube of the treatment device can be discharged out of the body through the flow path of the outlet portion.

In the connector for a treatment device according to the present disclosure, preferably, the first holding portion has a first sandwiching portion that sandwiches and fixes the first tube, and the second holding portion has a second sandwiching portion that sandwiches and fixes the second tube.

With the connector for a treatment device according to the present disclosure, the first sandwiching portion of the first holding portion sandwiches and fixes the first tube of the treatment device. The second sandwiching portion of the second holding portion sandwiches and fixes the second tube of the treatment device. Thereby, after the second tube of the treatment device is arranged in the inner cavity of the first tube of the treatment device and moved in the inner cavity of the first tube of the treatment device, the connector for a treatment device according to the present disclosure can fix positions of the first tube and the second tube of the treatment device. As a result, the connector for a treatment device according to the present disclosure can fix an injection position of the second tube of the treatment device and a discharge position of the first tube of the treatment device after adjustment.

In the connector for a treatment device according to the present disclosure, preferably, the first close contact portion has a portion whose inner diameter gradually decreases in a direction in which the first tube is inserted from the first insertion port, and the second close contact portion has a portion whose inner diameter gradually decreases in a direction in which the second tube is inserted from the second insertion port.

With the connector for a treatment device according to the present disclosure, the first close contact portion has the portion whose inner diameter gradually decreases in the direction in which the first tube of the treatment device is inserted from the first insertion port. Therefore, the first tube of the treatment device is reliably in close contact with the portion of the first close contact portion where the inner diameter gradually decreases. The second close contact portion also has the portion whose inner diameter gradually decreases in the direction in which the second tube of the treatment device is inserted from the second insertion port. Therefore, the second tube of the treatment device is reliably in close contact with the portion of the second close contact portion where the inner diameter gradually decreases. Therefore, it is possible to suppress generation of gaps between the first tube and the first close contact portion of the treatment device and between the second tube and the second close contact portion of the treatment device due to the negative pressure generated when the first tube of the treatment device draws the fluid present in the living body lumen and discharges the fluid out of the body of the patient. As a result, when the first tube of the treatment device draws the fluid present in the living body lumen and discharges the fluid out of the body of the patient, it is possible to suppress air from entering the inside of the first tube of the treatment device from the gap between the first tube and the first close contact portion of the treatment device and from the gap between the second tube and the second close contact portion of the treatment device, and it is possible to suppress a decrease in the efficiency of discharging the fluid.

A connector fixture for a treatment device according to the present disclosure, the connector fixture fixing a connector, connected to a treatment device including a tube to be inserted into a living body lumen of a patient, to a body surface of the patient, the connector fixture including: a body surface attachment portion attached to the body surface of the patient; a tube holding portion that holds the tube; and a connector attachment portion to which the connector is attached, the tube holding portion having a curved portion provided at a portion with which the tube comes into contact, and the connector attachment portion being capable of adjusting an attachment position of the connector along an axial direction of the tube.

With the connector fixture for a treatment device according to the present disclosure, the body surface attachment portion is attached to the body surface of the patient. The tube holding portion holds the tube of the treatment device to be inserted into the living body lumen of the patient. The connector attachment portion allows attachment of the connector to be connected to the treatment device. Here, the connector attachment portion can adjust the attachment position of the connector along the axial direction of the tube of the treatment device. Therefore, the connector fixture for a treatment device according to the present disclosure can be fixed to the body surface of the patient at a free position within a certain range along the axial direction of the tube of the treatment device from a position (that is, a puncture site) where the tube of the treatment device is inserted into the living body lumen of the patient regardless of a length by which the tube of the treatment device is inserted into the living body lumen of the patient, and can suppress the tube of the treatment device from being kinked or dropping out. Furthermore, the tube holding portion has the curved portion provided at the portion with which the tube of the treatment device comes into contact. Therefore, it is possible to prevent the tube of the treatment device from being kinked in the vicinity of the puncture site.

A fluid circulation system configured to inject a fluid into a subarachnoid space and to discharge the fluid in the subarachnoid space from a body of a patient, the fluid circulation system comprising: a catheter system including a first tube and a second tube, the second tube having an outer diameter smaller than an inner diameter of the first tube and is configured to be arranged in an inner cavity of the first tube, the first tube is configured to be inserted into the subarachnoid space of the patient and to discharge the fluid present in the subarachnoid space out of a body of the patient through a space between the inner cavity of the first tube and an outer side of the second tube; an oxygen supply device configured to oxygenate the fluid; and wherein the fluid including cerebrospinal fluid is configured to be discharged from the subarachnoid space of the patient through the inner cavity of the first tube and the oxygenated fluid is injected through the second tube into the subarachnoid space.

A method for treating a brain disease comprising: arranging a second tube in an inner cavity of a first tube, the second tube being movable in the inner cavity of the first tube such that an end portion of the second tube is exposed from an end portion of the first tube in a longitudinal direction of the first tube; inserting the first tube into a subarachnoid space of a patient and discharging cerebrospinal fluid present in the subarachnoid space out of a body of the patient through a space between the inner cavity of the first tube and an outer side of the second tube; and inserting the second tube into the subarachnoid space and injecting a fluid which includes oxygen higher in concentration than the cerebrospinal fluid through an inner cavity of the second tube into the cerebrospinal fluid present in the subarachnoid space.

According to the present disclosure, it is possible to provide the brain disease treatment device capable of performing injection and discharge at one puncture site and capable of independently adjusting the injection position and the discharge position after being inserted into the living body, the connector for a treatment device connected to the treatment device, and the connector fixture for a treatment device for fixing the connector for a treatment device to the body surface of the patient.

### BRIEF DESCRIPTION OF THE DRAWINGS

FIG. 1 is a block diagram illustrating an outline of a brain disease treatment system in which a brain disease treatment device according to the present embodiment is used.

FIG. 2 is a schematic view illustrating the brain disease treatment device according to the present embodiment.

FIG. 3 is a plan view illustrating a distal portion of the brain disease treatment device according to the present embodiment.

FIG. 4 is a plan view illustrating a proximal portion of the brain disease treatment device according to the present embodiment.

FIG. 5 is a cross-sectional view taken along a cutting plane A23-A23 illustrated in FIG. 4.

FIG. 6 is a perspective view illustrating a specific example of a first tube of the present embodiment.

FIG. 7 is a plan view illustrating the first tube of the specific example.

FIG. 8 is a cross-sectional view illustrating a connector for a treatment device according to the present embodiment.

FIG. 9 is a perspective view illustrating a specific example of the connector fixture for a treatment device according to the present embodiment and the connector for a treatment device according to the present embodiment.

### DETAILED DESCRIPTION

Hereinafter, a preferred embodiment of the present disclosure will be described in detail with reference to the drawings in a form including technical contents (an operation method and an operation/handling procedure).

Note that the embodiment described below is a preferred specific example of the invention, and thus various technically preferable limitations are given. However, the scope of the invention is not limited to these aspects unless there is a description to limit the invention in the following description. Furthermore, in the drawings, similar components are denoted by the same reference signs, and the detailed description thereof will be appropriately omitted.

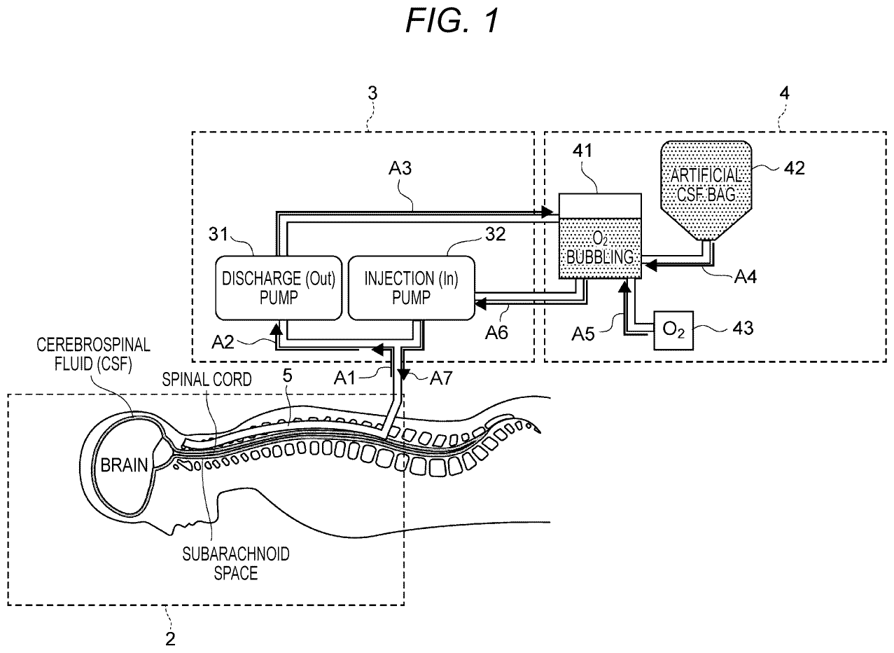

FIG. 1 is a block diagram illustrating an outline of a brain disease treatment system in which a brain disease treatment device according to the present embodiment is used.

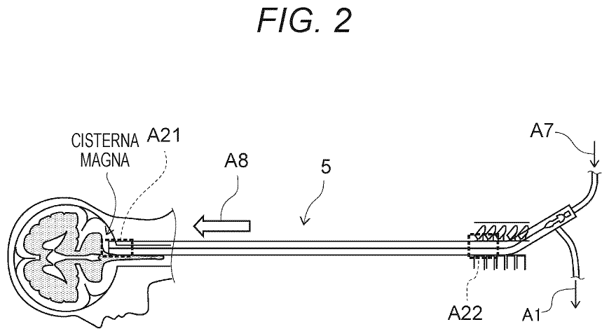

FIG. 2 is a schematic view illustrating the brain disease treatment device according to the present embodiment.

As illustrated in FIG. 1, the brain disease treatment system in which a brain disease treatment device 5 according to the present embodiment is used includes a spinal subarachnoid space 2, a pump system 3, and an oxygen supply device 4. The brain disease treatment device 5 is used in the spinal subarachnoid space catheter system 2.

The brain disease treatment device 5 is inserted into a subarachnoid space from the vicinity of a lumbar vertebrae in a lateral decubitus state and delivered to the vicinity of the cisterna magna, injects a high oxygen solution (in the present embodiment, oxygenated cerebrospinal fluid) at a distal portion into cerebrospinal fluid (CSF) present in the subarachnoid space, and draws the cerebrospinal fluid present in the subarachnoid space at a proximal portion and discharges the cerebrospinal fluid out of a body of a patient. The high oxygen solution of the present embodiment is an example of a “fluid” of the present disclosure.

The pump system 3 can include a discharge pump 31 and an injection pump 32. As indicated by arrows A1, A2, and A3 in FIG. 1, the discharge pump 31 draws the cerebrospinal fluid through the brain disease treatment device 5 and sends the cerebrospinal fluid to the oxygen supply device 4. A flow rate when drawing (that is, discharging) the cerebrospinal fluid from the subarachnoid space can be, for example, about 0.1 mL/min or more and 500 mL/min or less (i.e., 0.1 mL/min to 500 mL/min). However, the drawing flow rate (that is, the discharge flow rate) is not limited to 0.1 mL/min or more and 500 mL/min or less.

As indicated by arrows A6 and A7 in FIG. 1, the injection pump 32 draws the high oxygen solution supplied from the oxygen supply device 4 and injects the high oxygen solution into the cerebrospinal fluid through the brain disease treatment device 5. A flow rate when injecting the high oxygen solution into the cerebrospinal fluid can be, for example, about 0.1 mL/min or more and 500 mL/min or less (i.e., 0.1 mL/min to 500 mL/min). However, the injection rate is not limited to 0.1 mL/min or more and 500 mL/min or less.

In addition to a method of using two pumps of the discharge pump 31 and the injection pump 32 as in the present embodiment, the injection and discharge may be performed by a method of performing injection and discharge using one pump, a method of performing natural discharge using a pump only for injection, a method of performing natural injection using a pump only for discharge, or a method of performing injection and discharge using potential energy like drip without using a pump.

The oxygen supply device 4 can include oxygen bubbling 41, an artificial cerebrospinal fluid bag 42, and an oxygen supply source 43. The artificial cerebrospinal fluid bag 42 stores artificial cerebrospinal fluid (aCSF), and supplies the artificial cerebrospinal fluid to the oxygen bubbling 41 as indicated by an arrow A4 illustrated in FIG. 1. The artificial cerebrospinal fluid can be formed using, for example, a fluid containing lactated Ringer's solution. The oxygen supply source 43 supplies oxygen to the oxygen bubbling 41 as indicated by an arrow A5 in FIG. 1. The oxygen bubbling 41 mixes the cerebrospinal fluid supplied from the discharge pump 31, the artificial cerebrospinal fluid supplied from the artificial cerebrospinal fluid bag 42, and the oxygen supplied from the oxygen supply source 43 to generate an oxygenated cerebrospinal fluid, and supplies the oxygenated cerebrospinal fluid as the high oxygen solution to the injection pump 32 as indicated by an arrow A6 in FIG. 1.

Although the oxygen supply device 4 uses the oxygen bubbling 41 in the present embodiment, a hollow fiber may be immersed in cerebrospinal fluid and oxygen may be caused to pass through the hollow fiber, thereby oxygenating the cerebrospinal fluid through pores on the surface of the hollow fiber.

As illustrated in FIG. 2, the brain disease treatment device 5 is inserted into the subarachnoid space from the vicinity of the lumbar vertebrae of the patient. The distal portion of the brain disease treatment device 5 is delivered to the vicinity of the cisterna magna of the patient. Then, as indicated by an arrow A8 in FIG. 2, the high oxygen solution is sent to the distal portion of the brain disease treatment device 5, and is injected into the cerebrospinal fluid present in the subarachnoid space. An arrow A1 illustrated in FIG. 2 corresponds to the arrow A1 illustrated in FIG. 1. An arrow A7 illustrated in FIG. 2 corresponds to the arrow A7 illustrated in FIG. 1.

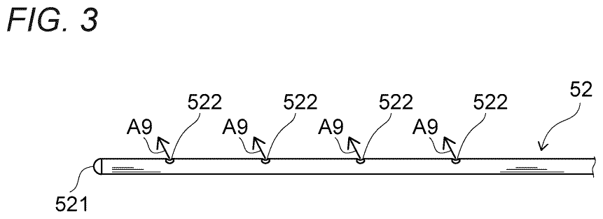

FIG. 3 is a plan view illustrating the distal portion of the brain disease treatment device according to the present embodiment.

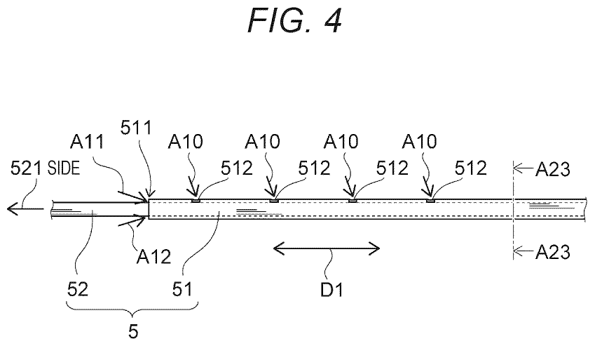

FIG. 4 is a plan view illustrating the proximal portion of the brain disease treatment device according to the present embodiment.

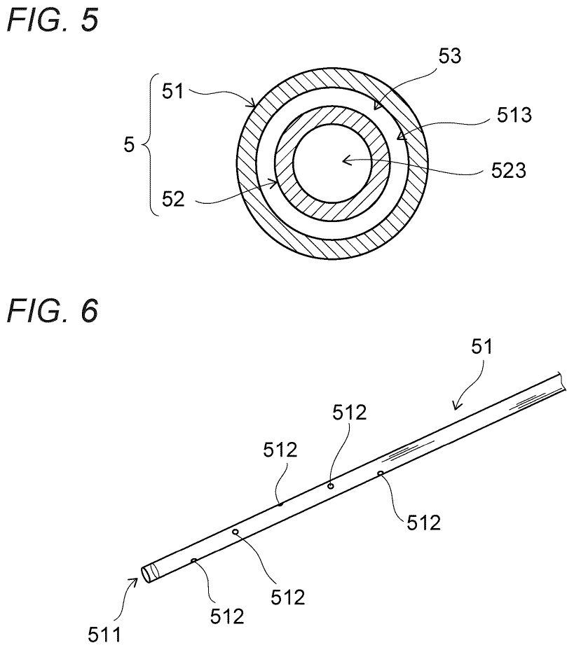

FIG. 5 is a cross-sectional view taken along a cutting plane A23-A23 illustrated in FIG. 4.

FIG. 3 is an enlarged view illustrating the brain disease treatment device 5 in a region A21 illustrated in FIG. 2 in an enlarged manner. FIG. 4 is an enlarged view illustrating the brain disease treatment device 5 in a region A22 illustrated in FIG. 2 in an enlarged manner.

As illustrated in FIG. 4, the brain disease treatment device 5 has a first tube 51 and a second tube 52.

The first tube 51 has a plurality of holes 512 on a side surface of the first tube 51. The plurality of holes 512 allow an inner cavity 513 (see FIG. 5) of the first tube 51 to communicate with the outer side of the first tube 51. A distal portion 511 of the first tube 51 is open and arranged in the subarachnoid space in the vicinity of the lumbar vertebrae. As indicated by arrows A10, A11, and A12 in FIG. 4, the first tube 51 is inserted into the subarachnoid space of the patient and draws the cerebrospinal fluid present in the subarachnoid space in the vicinity of the lumbar vertebrae into a space 53 (see FIG. 5) between the inner cavity 513 of the first tube 51 and the outer side of the second tube 52 through the opening in the distal portion 511 and the plurality of holes 512. Note that a force for drawing the cerebrospinal fluid is given by the discharge pump 31 as described above with respect to FIG. 1. Then, the first tube 51 discharges the cerebrospinal fluid out of the body of the patient through the space 53 as indicated by the arrow A1 in FIGS. 1 and 2.

An outer diameter of the second tube 52 is smaller than an inner diameter of the first tube 51. The second tube 52 can be arranged in the inner cavity 513 of the first tube 51. Furthermore, the second tube 52 is not connected to the first tube 51 and is movable within the inner cavity 513 of the first tube 51 along a longitudinal direction D1 (see FIG. 4) of the first tube 51. Since the distal portion 511 of the first tube 51 is open, a distal portion 521 of the second tube 52 can pass through the opening of the distal portion 511 of the first tube 51 as illustrated in FIG. 4.

As a result, the distal portion 521 of the second tube 52 can be exposed from the distal portion 511 of the first tube 51 in the longitudinal direction D1 of the first tube 51. A distance in the longitudinal direction D1 between the distal portion 511 of the first tube 51 and the distal portion 521 of the second tube 52 exposed from the distal portion 511 of the first tube 51 can be adjusted to a predetermined distance. The “predetermined distance” in the specification of the present application can be, for example, about 0 cm or more and 30 cm or less (i.e., 0 cm to 30 cm). If the distance between the distal portion 521 of the second tube 52 and the distal portion 511 of the first tube 51 is too short, the high oxygen solution injected into the cerebrospinal fluid through the second tube 52 is drawn by the first tube, and an effect of the high oxygen solution injection is reduced. Therefore, a maximum adjustable distance between the distal portion 511 of the first tube 51 and the distal portion 521 of the second tube 52 is preferably set to 10 cm or more. Furthermore, a distance from the cisterna magna assumed as a placement position of the distal portion 521 of the second tube 52 to a position on the head side at several centimeters from a lumbar vertebrae puncture portion assumed as a placement position of the distal portion 511 of the first tube 51 is considered to be 30 cm to 50 cm. Therefore, it is more preferable if the maximum adjustable distance between the distal portion 511 of the first tube 51 and the distal portion 521 of the second tube 52 is 30 cm or more since the brain disease treatment device 5 can cope with various technical strategies in consideration of a body shape of the patient, a treatment effect, a risk at the time of delivery of the second tube 52, and the like. The maximum adjustable distance between the distal portion 511 of the first tube 51 and the distal portion 521 of the second tube 52 can include, for example, a case where the distance between the distal portion 511 of the first tube 51 and the distal portion 521 of the second tube 52 can be adjusted to 0 cm to 30 cm. Furthermore, it is considered that the brain disease treatment device 5 can be applied to most patients if the maximum adjustable distance is 50 cm. However, the “predetermined distance” in the specification of the present application is not limited to 0 cm or more and 30 cm or less (i.e., 0 cm to 30 cm).

The second tube 52 has a plurality of holes 522 on a side surface of the second tube 52. The holes 522 allow an inner cavity 523 (see FIG. 5) of the second tube 52 to communicate with the outer side of the second tube 52. The distal portion 521 of the second tube 52 is closed and is arranged in the vicinity of the cisterna magna through the opening of the distal portion 511 of the first tube 51. As indicated by an arrow A9 in FIG. 3, the second tube 52 is inserted into the subarachnoid space of the patient and injects the high oxygen solution through the inner cavity 523 and the plurality of holes 522 of the second tube 52 into the cerebrospinal fluid present in the subarachnoid space in the vicinity of the cisterna magna. Note that a force for injecting the high oxygen solution into the cerebrospinal fluid is given by the injection pump 32 as described above with reference to FIG. 1.

Since the distal portion 521 of the second tube 52 is closed, it is considered that the second tube 52 can diffuse the high oxygen solution in all directions through the plurality of holes 522 about an injection point with respect to a wide space of the cisterna magna. Although the configuration in which the distal portion 521 of the second tube 52 is closed has been described in the present embodiment, a configuration in which the distal portion 521 of the second tube 52 is open may be adopted. For example, if the configuration in which the distal portion 521 of the second tube 52 is open is adopted in a case where the space into which the high oxygen solution is injected is not wide, the second tube 52 can inject the high oxygen solution forward to make the high oxygen solution reach the deeper side.

In the cutting plane A23-A23 (see FIG. 5) in a direction perpendicular to the longitudinal direction D1, a cross-sectional area of the space 53 between the outer side of the second tube 52 and the inner side of the first tube 51 is set within a predetermined ratio with respect to a cross-sectional area of the inner cavity 523 of the second tube 52 in order to keep ICP (intracranial pressure) constant within a certain range. Since it is not preferable that the ICP becomes higher or lower than a limit range, it is preferable to set a ratio of the cross-sectional area of the space 53 with respect to the cross-sectional area of the inner cavity 523 within a certain range around 1 time. The “predetermined ratio” in the specification of the present application is preferably, for example, about 0.5 times or more and 2 times or less (i.e., 0.5 time to 2.0 times). However, the “predetermined ratio” in the specification of the present application is not limited to 0.5 times or more and 2 times or less.

Hereinafter, a first specific example of a treatment method using the brain disease treatment device 5 of the present embodiment will be described with reference to FIGS. 1 and 4.

First, a puncture needle of a puncture device is stuck from a lumbar vertebrae of a patient to a subarachnoid space. Thereafter, the first tube 51 is inserted into an inner cavity of the puncture needle of the puncture device and inserted into the subarachnoid space, and the distal portion 511 of the first tube 51 is placed at a position on the head side at several centimeters from a lumbar vertebrae puncture site. Thereafter, the puncture needle is removed from the lumbar vertebrae, and a proximal portion of the first tube 51 is inserted into a first insertion port 612 of a connector 6 for a treatment device and fixed. Thereafter, the second tube 52 is inserted into a second insertion port 622 of the connector 6 for a treatment device to insert the second tube 52 into the inner cavity of the first tube 51, and the distal portion 521 of the second tube 52 is delivered to the head side of the subarachnoid space and placed in the vicinity of the cisterna magna. Then, at the same time as the high oxygen solution is injected into cerebrospinal fluid in the cisterna magna of the subarachnoid space through the holes 522 of the second tube 52 by operating the injection pump 32 connected to the proximal side of the inner cavity of the second tube 52, the discharge pump 31 connected to the proximal side of the inner cavity of the first tube 51 is operated to discharge the cerebrospinal fluid out of a body through the opening of the distal portion 511 and the holes 512 of the first tube 51 in an amount substantially equal to an amount of the injected high oxygen solution in order to keep the intracranial pressure within an appropriate range. As a result, a concentration of oxygen of the cerebrospinal fluid increases, and oxygen is supplied to a region of the brain where oxygen is insufficient to prevent or mitigate the progress of cerebral infarction. The first tube 51 and the second tube 52 are removed from the lumbar vertebrae of the patient when treatment is finished.

A second specific example of the treatment method using the brain disease treatment device 5 of the present embodiment will be described.

First, a puncture needle of a puncture device is stuck from a lumbar vertebrae of a patient to a subarachnoid space. Thereafter, a guide wire is inserted into an inner cavity of the puncture needle of the puncture device and inserted into the subarachnoid space. Thereafter, only the puncture device is removed from the lumbar vertebrae while maintaining a state where a guide wire is inserted into the subarachnoid space. Then, only the guide wire is stuck from the lumbar vertebrae to the subarachnoid space. Thereafter, the guide wire is put into the inner cavity of the first tube 51, the first tube 51 is advanced along the guide wire, and the first tube 51 is inserted into the subarachnoid space. Thereafter, only the guide wire is removed while maintaining the state in which the first tube 51 is inserted from the lumbar vertebrae to the subarachnoid space. Then, only the first tube 51 is inserted from the lumbar vertebrae to the subarachnoid space. Thereafter, the second tube 52 is inserted into the inner cavity of the first tube 51, and the distal portion 521 of the second tube 52 is delivered to the head side of the subarachnoid space and placed in the vicinity of the cisterna magna. Then, at the same time as the high oxygen solution is injected into cerebrospinal fluid in the cisterna magna of the subarachnoid space through the holes 522 of the second tube 52 by operating the injection pump 32 connected to the proximal side of the inner cavity of the second tube 52, the discharge pump 31 connected to the proximal side of the inner cavity of the first tube 51 is operated to discharge the cerebrospinal fluid out of a body through the opening of the distal portion 511 and the holes 512 of the first tube 51 in an amount substantially equal to an amount of the injected high oxygen solution in order to keep the intracranial pressure within an appropriate range. As a result, a concentration of oxygen of the cerebrospinal fluid increases, and oxygen is supplied to a region of the brain where oxygen is insufficient to prevent or mitigate the progress of cerebral infarction. The first tube 51 and the second tube 52 are removed from the lumbar vertebrae of the patient when treatment is finished.

Although the example in which the first tube 51 and the second tube 52 are inserted from the lumbar vertebrae of the patient to the subarachnoid space has been described in the first specific example and the second specific example of the above treatment method, the first tube 51 and the second tube 52 may be inserted from the head of the patient to the subarachnoid space.

Furthermore, the example in which the injection of the high oxygen solution and the discharge of the cerebrospinal fluid are performed using the two pumps of the injection pump 32 and the discharge pump 31 has been described in the above description, but the injection of the high oxygen solution and the discharge of the cerebrospinal fluid may be performed using one pump. Alternatively, instead of the high oxygen solution or together with the high oxygen solution, the cerebrospinal fluid discharged from the subarachnoid space of the patient may be oxygenated by the oxygen supply device 4 and injected into the subarachnoid space of the patient. With the brain disease treatment device 5 according to the present

embodiment, after the first tube 51 and the second tube 52 are inserted into the subarachnoid space from one puncture site in the vicinity of the lumbar vertebrae, the second tube 52 can move in the inner cavity 513 of the first tube 51 and can be exposed from the distal portion 511 of the first tube 51 in the longitudinal direction D1. As a result, the brain disease treatment device 5 according to the present embodiment can perform the injection of the high oxygen solution into the cerebrospinal fluid and the discharge of the cerebrospinal fluid at one puncture site, and can independently adjust an injection position of the second tube 52 for injecting the high oxygen solution into the cerebrospinal fluid and a discharge position of the first tube 51 for discharging the cerebrospinal fluid out of the body of the patient. As a result, when cerebral infarction has occurred, it is possible to adopt an arrangement capable of efficiently delivering the high oxygen solution according to the body shape of the patient and a region where the cerebral infarction has occurred, and thus, damage to brain cells can be suppressed. Furthermore, when the cerebral infarction has occurred, it is possible to appropriately arrange the tubes in accordance with a location of the occurrence, and it is possible to efficiently administer the high oxygen solution to suppress the damage to the brain cells.

Furthermore, in the brain disease treatment device 5 according to the present embodiment, the distance in the longitudinal direction D1 between the distal portion 511 of the first tube 51 and the distal portion 521 of the second tube 52 exposed from the distal portion 511 of the first tube 51 can be adjusted to the predetermined distance or more. Therefore, the discharge position of the first tube 51 and the injection position of the second tube 52 can be separated by a predetermined distance or more in the longitudinal direction D1. Therefore, it is possible to prevent the high oxygen solution injected into the cerebrospinal fluid by the second tube 52 from being immediately drawn by the first tube 51 and discharged out of the body of the patient, which makes it possible to efficiently discharge the cerebrospinal fluid and inject the high oxygen solution into the cerebrospinal fluid.

Since the cross-sectional area of the space 53 is within the range of the predetermined ratio with respect to the cross-sectional area of the inner cavity 523 of the second tube 52 in the cutting plane A23-A23 in the direction perpendicular to the longitudinal direction D1, the discharge of the cerebrospinal fluid and the injection can be efficiently performed while keeping the balance between the discharge of the cerebrospinal fluid and the injection of the fluid into the cerebrospinal fluid.

FIG. 6 is a perspective view illustrating a specific example of the first tube of the present embodiment.

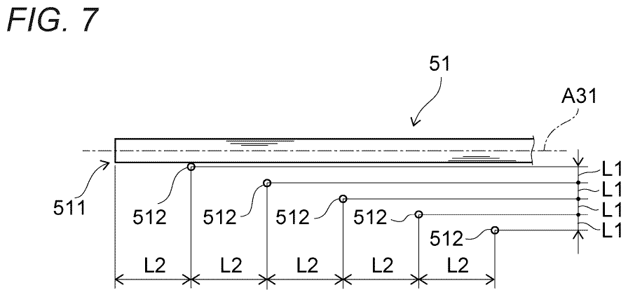

FIG. 7 is a plan view illustrating the first tube of the specific example.

FIG. 7 schematically illustrates a state in which each of the holes 512 appearing when the first tube 51 is rotated in the circumferential direction about an axis A31 of the first tube 51 is projected on a predetermined plane.

As illustrated in FIG. 6, the plurality of holes 512 are arranged in a spiral shape on the side surface of the first tube 51. More specifically, as illustrated in FIG. 7, the plurality of holes 512 are provided at positions different from each other in the circumferential direction of the first tube 51 and separated from each other at substantially equal intervals L1. Further, the plurality of holes 512 are provided at positions different from each other in the longitudinal direction D1 (that is, a direction of the axis A31) and separated from each other at substantially equal intervals L2. Note that a direction of the spiral may be either a rightward or leftward winding direction toward a distal direction.

Since the plurality of holes 512 are arranged in the spiral shape on the side surface of the first tube 51 in this manner, it is possible to prevent all of the plurality of holes 512 from being blocked by tissue even in a case where the tissue in a living body of the patient adheres to any one of the plurality of holes 512 when the first tube 51 is inserted into the subarachnoid space to draw the cerebrospinal fluid. As a result, it is possible to prevent the first tube 51 from being unable to draw and discharge the cerebrospinal fluid.

Although a case where the plurality of holes 512 are arranged in the spiral shape is exemplified in FIGS. 6 and 7, an arrangement form of the plurality of holes 512 is not limited to the spiral shape. For example, the plurality of holes 512 may be randomly arranged in the longitudinal direction D1 and the circumferential direction.

Alternatively, for example, as illustrated in FIG. 4, the plurality of holes 512 may be provided at the same position in the circumferential direction of the first tube 51 and at positions different from each other in the longitudinal direction D1 and separated from each other at the substantially equal intervals L2. Alternatively, the plurality of holes 512 may be provided at the same position in the longitudinal direction D1 and at positions different from each other in the circumferential direction of the first tube 51 and separated from each other at the substantially equal intervals L1. Even in such cases, it is possible to prevent all of the plurality of holes 512 from being blocked by the tissue and to prevent the first tube 51 from being unable to draw and discharge the cerebrospinal fluid as in the case where the plurality of holes 512 are arranged in the spiral shape.

Next, a connector for a treatment device according to the present embodiment will be described with reference to the drawings.

In the description of the present embodiment, a case where a treatment device connected to the connector 6 for a treatment device is the brain disease treatment device 5 described above with reference to FIGS. 1 to 7 will be described as an example. Therefore, the description of the treatment device connected to the connector 6 for a treatment device is appropriately omitted, and the connector 6 for a treatment device will be mainly described hereinafter.

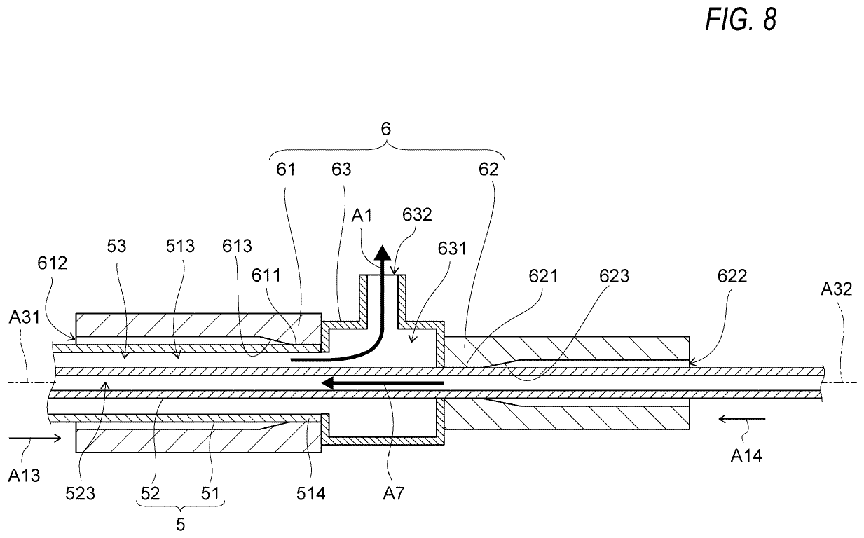

FIG. 8 is a cross-sectional view illustrating the connector for a treatment device according to the present embodiment.

The connector 6 for a treatment device includes a first holding portion 61, a second holding portion 62, and an outlet portion 63.

The first holding portion 61 has a first close contact portion 611. An inner diameter of the first close contact portion 611 is equal to or smaller than an outer diameter of the first tube 51. The first holding portion 61 holds the first tube 51, inserted from the first insertion port 612 in a direction of an arrow A13 illustrated in FIG. 8, at the first close contact portion 611. The first close contact portion 611 has a first tapered portion 613. The first tapered portion 613 is a portion whose inner diameter gradually decreases in a direction in which the first tube 51 is inserted from the first insertion port 612 (that is, the direction of the arrow A13). Since the first close contact portion 611 has the first tapered portion 613, the first tube 51 is reliably brought into close contact with the first tapered portion 613 of the first close contact portion 611 in a liquid-tight manner. As a result, the first tube 51 can be fixed inside the first holding portion 61.

The second holding portion 62 has a second close contact portion 621. An inner diameter of the second close contact portion 621 is equal to or smaller than the outer diameter of the second tube 52. The second holding portion 62 holds the second tube 52, inserted from the second insertion port 622 in a direction of an arrow A14 illustrated in FIG. 8, at the second close contact portion 621. The second close contact portion 621 has a second tapered portion 623. The second tapered portion 623 is a portion whose inner diameter gradually decreases in a direction in which the second tube 52 is inserted from the second insertion port 622 (that is, the direction of the arrow A14). Since the second close contact portion 621 has the second tapered portion 623, the second tube 52 is reliably brought into close contact with the second tapered portion 623 of the second close contact portion 621 in a liquid-tight manner.

The outlet portion 63 is sandwiched between the first holding portion 61 and the second holding portion 62, and is fixed to the first holding portion 61 and the second holding portion 62. The first holding portion 61, the second holding portion 62, and the outlet portion 63 may be integrally formed, or may be separately formed and joined to each other.

The outlet portion 63 has a flow path 631 through which the cerebrospinal fluid passing through the space 53 flows. As described above with respect to FIGS. 3 to 5, the cerebrospinal fluid passing through the space 53 is the cerebrospinal fluid drawn into the space 53 through the opening of the distal portion 511 and the plurality of holes 512. As indicated by an arrow A1 in FIG. 8, the cerebrospinal fluid flowing through the flow path 631 is discharged from an outlet 632 provided in the outlet portion 63. The arrow A1 illustrated in FIG. 8 corresponds to the arrow A1 illustrated in FIGS. 1 and 2.

When viewed along a direction of an axis A31 of the first tube 51 held by the first holding portion 61 or a direction of an axis A32 of the second tube 52 held by the second holding portion 62, the second close contact portion 621 is present on the inner side of the first close contact portion 611. As illustrated in FIG. 8, the second tube 52 inserted from the second insertion port 622 can be inserted into the inner cavity 513 of the first tube 51 fixed to the first holding portion 61 and can penetrate the first tube 51. Then, the high oxygen solution is injected into the cerebrospinal fluid present in the subarachnoid space in the vicinity of the cisterna magna through the inner cavity 523 of the second tube 52 as indicated by an arrow A7 illustrated in FIG. 8. The arrow A7 illustrated in FIG. 8 corresponds to the arrow A7 illustrated in FIGS. 1 and 2. On the other hand, the cerebrospinal fluid discharged from a proximal portion 514 of the first tube 51 fixed to the first holding portion 61 passes through the flow path 631 of the outlet portion 63 and is discharged from the outlet 632 as indicated by the arrow A1 illustrated in FIG. 8.

With the connector 6 for a treatment device according to the present embodiment, for example, after the first tube 51 of the brain disease treatment device 5 is inserted into the living body of the patient through a spinal needle stuck into the living body of the patient and the spinal needle is removed, the first holding portion 61 can hold the first tube 51 inserted from the first insertion port 612 at the first close contact portion 611. Furthermore, the second holding portion 62 can hold the second tube 52 inserted from the second insertion port 622 at the second close contact portion 621. Here, when viewed along the direction of the axis A31 of the first tube 51 held by the first holding portion 61 or the direction of the axis A32 of the second tube 52 held by the second holding portion 62, the second close contact portion 621 is present on the inner side of the first close contact portion 611. Therefore, the second tube 52 can be easily arranged in the inner cavity 513 of the first tube 51 through the second close contact portion 621 without alignment and easily moved in the longitudinal direction (axial direction) in the inner cavity 513 of the first tube 51. The outlet portion 63 has the flow path 631 through which the cerebrospinal fluid passing through the space 53 flows and the outlet 632 of the flow path 631. In this manner, when the first tube 51 is inserted into the living body of the patient using, for example, a spinal needle, the connector 6 for a treatment device according to the present embodiment can be mounted on the first tube 51 after the spinal needle is removed. Then, after the connector 6 for a treatment device according to the present embodiment is mounted on the first tube 51, the second tube 52 can be easily arranged in the inner cavity 513 of the first tube 51 and easily moved in inner cavity 513 of the first tube 51 in the longitudinal direction (axial direction).

Furthermore, when the first tube 51 is inserted into the living body of the patient using, for example, a spinal needle, the connector 6 for a treatment device according to the present embodiment is mounted on the first tube 51 and the second tube 52 after the spinal needle is removed, so that the cerebrospinal fluid discharged from the proximal portion 514 of the first tube 51 can be discharged out of the body through the flow path 631 of the outlet portion 63.

Furthermore, in the connector 6 for a treatment device according to the present embodiment, the first tube 51 is reliably in close contact with the first tapered portion 613 of the first close contact portion 611 in a liquid-tight manner as described above. The second tube 52 is reliably in close contact with the second tapered portion 623 of the second close contact portion 621 in a liquid-tight manner.

Therefore, it is possible to suppress generation of gaps between the first tube 51 and the first close contact portion 611 and between the second tube 52 and the second close contact portion 621 due to the negative pressure generated when the first tube 51 draws the cerebrospinal fluid and discharges the cerebrospinal fluid out of the body of the patient. As a result, when the first tube 51 draws the cerebrospinal fluid and discharges the cerebrospinal fluid out of the body of the patient, it is possible to suppress air from entering the inside of the first tube 51 from the gap between the first tube 51 and the first close contact portion 611 and the gap between the second tube 52 and the second close contact portion 621, and to suppress a decrease in the efficiency of discharging the cerebrospinal fluid.

Next, a specific example of a connector fixture for a treatment device according to the present embodiment and the connector for a treatment device according to the present embodiment will be described with reference to the drawings.

In the description of the present embodiment, a case where a connector that is fixed to the body surface of the patient by the connector fixture 7 for a treatment device attached to an appropriate site on the body surface of the patient is the connector 6 for a treatment device described above with reference to FIG. 8 will be described as an example. Furthermore, a case where a treatment device connected to the connector 6 for a treatment device is the brain disease treatment device 5 described above with reference to FIGS. 1 to 7 will be described as an example. Therefore, the description regarding the connector that is fixed to the body surface of the patient by the connector fixture 7 for a treatment device and the treatment device connected to the connector 6 for a treatment device will be appropriately omitted, and the connector fixture 7 for a treatment device will be mainly described hereinafter.

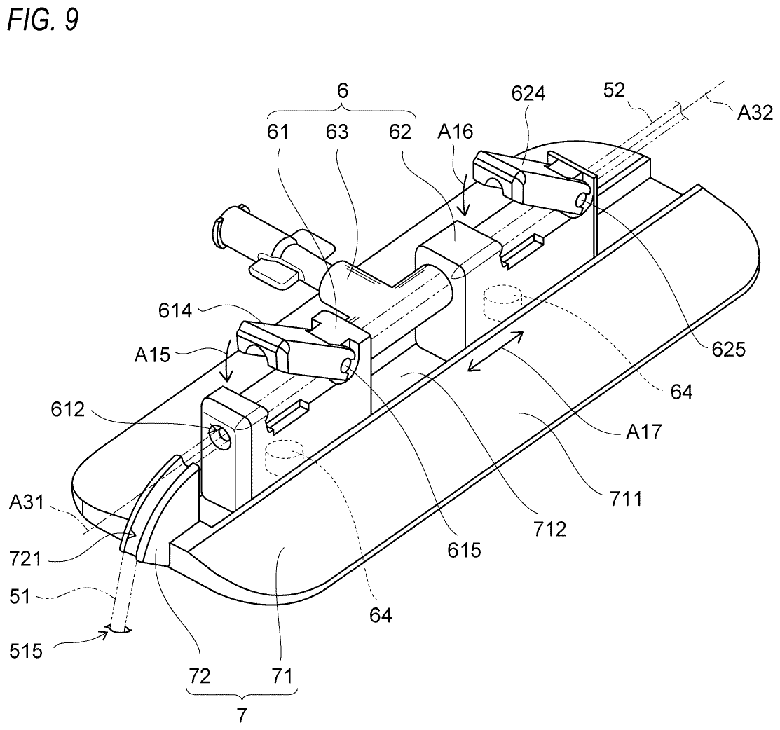

FIG. 9 is a perspective view illustrating a specific example of the connector fixture for a treatment device according to the present embodiment and the connector for a treatment device according to the present embodiment.

The connector fixture 7 for a treatment device includes a fixture body 71 and a tube holding portion 72.

The fixture body 71 includes a body surface attachment portion 711 and a connector attachment portion 712. As illustrated in FIG. 9, the body surface attachment portion 711 has a plate shape and is attached to the body surface of the patient. The connector attachment portion 712 allows the connector 6 for a treatment device to be attached to the fixture body 71. In the connector fixture 7 for a treatment device illustrated in FIG. 9, the connector attachment portion 712 is a groove formed along the longitudinal direction D1 (see FIG. 4) in the fixture body 71.

For example, as illustrated in FIG. 9, a magnet 64 is provided on a lower surface of each of the first holding portion 61 and the second holding portion 62. The lower surface of each of the first holding portion 61 and the second holding portion 62 is a surface facing the connector attachment portion 712. Since the magnets 64 are attracted to the connector attachment portion 712, the connector 6 for a treatment device is attached to the connector attachment portion 712. Then, as indicated by an arrow A17 illustrated in FIG. 9, the connector attachment portion 712 can adjust an attachment position of the connector 6 for a treatment device along a direction of an axis A31 of the first tube 51 held by the first holding portion 61 and a direction of an axis A32 of the second tube 52 held by the second holding portion 62.

As illustrated in FIG. 9, the tube holding portion 72 is fixed to a distal portion of the fixture body 71 and can hold the first tube 51. For example, a groove having substantially the same diameter as the outer diameter of the first tube 51 is formed in a portion where the tube holding portion 72 holds the first tube 51. The tube holding portion 72 has a curved portion 721 provided at a portion with which the first tube 51 comes into contact. As a result, the tube holding portion 72 can suppress the first tube 51 from being kinked (folded or the like) in the vicinity of a puncture site 515.

According to the connector fixture 7 for a treatment device of the present embodiment, the connector attachment portion 712 can adjust the attachment position of the connector 6 for a treatment device along the direction of the axis A31 of the first tube 51 held by the first holding portion 61 and the direction of the axis A32 of the second tube 52 held by the second holding portion 62. Therefore, regardless of a length by which the first tube 51 is inserted into the subarachnoid space, the connector fixture 7 for a treatment device can be fixed to the body surface of the patient at a free position within a certain range along the direction of the axis A31 of the first tube 51 from the position (that is, the puncture site 515) at which the first tube 51 is inserted into the subarachnoid space, which can prevent the first tube 51 from being kinked or dropping out.

Furthermore, in the specific example of the connector 6 for a treatment device illustrated in FIG. 9, the first holding portion 61 has a first sandwiching portion 614. As indicated by an arrow A15 illustrated in FIG. 9, the first sandwiching portion 614 is rotatably supported by a first support shaft 615 provided in the first holding portion 61. The first sandwiching portion 614 rotates in a closing direction with respect to the first holding portion 61 to sandwich and fix the first tube 51. Furthermore, the second holding portion 62 includes a second sandwiching portion 624. As indicated by an arrow A16 illustrated in FIG. 9, the second sandwiching portion 624 is rotatably supported by a second support shaft 625 provided in the second holding portion 62. The second sandwiching portion 624 rotates in a closing direction with respect to the second holding portion 62 to sandwich and fix the second tube 52.

As a result, the connector 6 for a treatment device can fix positions of the first tube 51 and the second tube 52 after the second tube 52 is arranged in the inner cavity 513 of the first tube 51 and moved in the inner cavity 513 of the first tube 51. As a result, the connector 6 for a treatment device can fix the injection position of the high oxygen solution by the second tube 52 and the discharge position (that is, the drawing position) of the cerebrospinal fluid by the first tube 51 after adjustment.

The detailed description above describes embodiments of a brain disease treatment device used for treatment of a brain disease, a connector for a treatment device connected to the treatment device, and a connector fixture for a treatment device for fixing a connector for a treatment device to a body surface of a patient. However, the invention is not limited to the above-described embodiment, and various modifications can be made without departing from the scope of the claims. The configurations of the above-described embodiment can be partially omitted, or can be arbitrarily combined so as to be different from the above-described configurations.

What is claimed is:

1. A brain disease treatment device comprising:
a first tube;
a second tube which has an outer diameter smaller than an inner diameter of the first tube and is configured to be arranged in an inner cavity of the first tube and movable in the inner cavity of the first tube;
the first tube is configured to be inserted into a subarachnoid space of a patient and to discharge cerebrospinal fluid present in the subarachnoid space out of a body of the patient through a space between the inner cavity of the first tube and an outer side of the second tube;
the second tube is configured to be inserted into the subarachnoid space and to inject a fluid through an inner cavity of the second tube into the cerebrospinal fluid present in the subarachnoid space; and
an end portion of the second tube is exposable from an end portion of the first tube in a longitudinal direction of the first tube.

2. The brain disease treatment device according to claim 1, wherein
a distance in the longitudinal direction between the end portion of the first tube and the end portion of the second tube exposed from the end portion of the first tube is adjustable.

3. The brain disease treatment device according to claim 1, wherein
a cross-sectional area of the space between the inner cavity of the first tube and the outer side of the second tube is within a range of a predetermined ratio with respect to a cross-sectional area of the inner cavity of the second tube in a cross section in a direction perpendicular to the longitudinal direction.

4. The brain disease treatment device according to claim 1, wherein
the first tube has a plurality of holes on a side surface of the first tube, the plurality of holes allowing the inner cavity of the first tube to communicate with an outer side of the first tube to communicate with each other; and
the plurality of holes are provided at positions different from each other in a circumferential direction of the first tube and separated from each other at substantially equal intervals.

5. The brain disease treatment device according to claim 4, wherein
the plurality of holes are provided at positions different from each other in the longitudinal direction and separated from each other at substantially equal intervals.

6. The brain disease treatment device according to claim 1, wherein the fluid is a higher oxygen solution than the cerebrospinal fluid.

7. A fluid circulation system configured to inject a fluid into a subarachnoid space and to discharge the fluid in the subarachnoid space from a body of a patient, the fluid circulation system comprising:
a catheter system including a first tube and a second tube, the second tube having an outer diameter smaller than an inner diameter of the first tube and is configured to be arranged in an inner cavity of the first tube, the first tube is configured to be inserted into the subarachnoid space of the patient and to discharge the fluid present in the subarachnoid space out of a body of the patient through a space between the inner cavity of the first tube and an outer side of the second tube;
an oxygen supply device configured to oxygenate the fluid; and
wherein the fluid including cerebrospinal fluid is configured to be discharged from the subarachnoid space of the patient through the inner cavity of the first tube and the oxygenated fluid is injected through the second tube into the subarachnoid space.

8. The fluid circulation system according to claim 7, wherein the fluid circulation system comprises a pump system including at least one pump, the at least one pump configured to inject and/or discharge the fluid.

9. The fluid circulation system according to claim 7, wherein an end portion of the second tube is configured to be exposed from an end portion of the first tube in a longitudinal direction of the first tube.

10. The fluid circulation system according to claim 7, wherein the second tube is movable in the inner cavity of the first tube.

11. A connector for the fluid circulation system according to claim 7, the connector comprising:
a first holding portion that has a first close contact portion whose inner diameter is equal to or smaller than an outer diameter of the first tube, and the first holding portion configured to hold the first tube inserted from a first insertion port at the first close contact portion;
a second holding portion that has a second close contact portion whose inner diameter is equal to or smaller than the outer diameter of the second tube, and the second holding portion configured to hold the second tube inserted from a second insertion port at the second close contact portion;
an outlet portion that has a flow path through which a fluid passing through the space between the inner cavity of the first tube and the outer side of the second tube flows, and an outlet of the flow path; and
wherein the second close contact portion is on an inner side of the first close contact portion when viewed along an axial direction of either the first tube held by the first holding portion or the second tube held by the second holding portion.

12. The connector according to claim 11, wherein
the first tube is configured to be fixed inside the first holding portion;
the second tube inserted from the second insertion port is configured to be inserted into the inner cavity of the first tube fixed to the first holding portion and penetrates the first tube; and
the fluid discharged from a proximal portion of the first tube fixed to the first holding portion passes through the flow path.

13. The connector according to claim 11, wherein
the first holding portion has a first sandwiching portion configured to sandwich and fix the first tube; and
the second holding portion has a second sandwiching portion configured to sandwich and fix the second tube.

14. The connector according to claim 11, wherein
the first close contact portion has a portion whose inner diameter gradually decreases in a direction in which the first tube is inserted from the first insertion port; and
the second close contact portion has a portion whose inner diameter gradually decreases in a direction in which the second tube is inserted from the second insertion port.

15. A connector fixture for the fluid circulation system according to claim 7, wherein the connector fixture is configured to fix a connector, the connector being connected to the catheter system, and wherein the connector fixture comprises:
a body surface attachment portion attached to the body surface of the patient;
a tube holding portion configured to hold the first tube;
a connector attachment portion to which the connector is attached; and
wherein the tube holding portion has a curved portion provided at a portion with which the first tube comes into contact, and the connector attachment portion is configured to adjust an attachment position of the connector along an axial direction of the first tube.

16. A method for treating a brain disease comprising:
arranging a second tube in an inner cavity of a first tube, the second tube being movable in the inner cavity of the first tube such that an end portion of the second tube is exposed from an end portion of the first tube in a longitudinal direction of the first tube;
inserting the first tube into a subarachnoid space of a patient and discharging cerebrospinal fluid present in the subarachnoid space out of a body of the patient through a space between the inner cavity of the first tube and an outer side of the second tube; and
inserting the second tube into the subarachnoid space and injecting a fluid which includes oxygen higher in concentration than the cerebrospinal fluid through an inner cavity of the second tube into the cerebrospinal fluid present in the subarachnoid space.

17. The method according to claim 16, further comprising:
adjusting a distance in the longitudinal direction between the end portion of the first tube and the end portion of the second tube exposed from the end portion of the first tube such that a distal portion of the first tube is placed in the vicinity of a lumbar vertebrae puncture site, and a distal portion of the second tube is delivered and placed to a head side of the subarachnoid space.

18. The method according to claim 16, wherein
a cross-sectional area of the space between the inner cavity of the first tube and the outer side of the second tube is within a range of a predetermined ratio with respect to a cross-sectional area of the inner cavity of the second tube in a cross section in a direction perpendicular to the longitudinal direction.

19. The method according to claim 16, wherein
the first tube has a plurality of holes on a side surface of the first tube, the plurality of holes allowing the inner cavity of the first tube to communicate with an outer side of the first tube to communicate with each other; and
the plurality of holes are provided at positions different from each other in a circumferential direction of the first tube and separated from each other at substantially equal intervals.

20. The method according to claim 19, wherein
the plurality of holes are provided at positions different from each other in the longitudinal direction and separated from each other at substantially equal intervals.
